(19) United States
(12) Patent Application Publication
(10) Pub. No.: US 2012/0116338 A1
(43) Pub. Date: May 10, 2012

(54) Polypropylene Nonwoven Fibers And Fabrics And Methods For Making Same

(76) Inventors: William M. Ferry, Houston, TX (US); Smita Kacker, Annandale, NJ (US); Olivier J. Georjon, Houston, TX (US)

(21) Appl. No.: 13/140,654

(22) PCT Filed: Dec. 21, 2009
(86) PCT No.: PCT/US09/69042

§ 371 Date: Jul. 21, 2011

Related U.S. Application Data
(60) Provisional application No. 61/148,253, filed on Jan. 29, 2009.

(51) Int. Cl. A61L 15/24; B32B 27/32; D01D 5/10; D04H 13/00

(52) U.S. Cl. 604/372; 442/334; 442/1; 264/210.8; 264/175

(57) ABSTRACT

Nonwoven fabrics of fibers comprising one or more primary polypropylenes having a molecular weight distribution of less than 3.5 and a melt flow rate within the range from 5 to 500 dg/min, the fibers having an average diameter of less than 20 μm, or a denier (g/9000 m) of less than 2.0, thus forming propylene-based fabrics. The primary polypropylene is preferably a reactor grade polymer made using a single-site catalyst. In certain embodiments, the propylene-based fabrics disclosed herein have a MD Tensile Strength (WSP 110.4 (05)) of greater than 20 N/5 cm when calendered at a temperature within the range from 110 to 15O° C. Also in certain embodiments, the fabrics have a CD Tensile Strength (WSP 110.4 (05)) of greater than 10 N/5 cm when calendered at a temperature within the range from 110 to 15O° C. The fabrics are preferably meltspun, and in a particular embodiment are spunbond fabrics.

### PRIORITY CLAIM

This application claims the benefit of prior U.S. provisional application Ser. No. 61/148,253 filed Jan. 29, 2009 which is hereby incorporated by reference in its entirety.

### FIELD OF THE INVENTION

The disclosure relates to propylene-based fibers and nonwoven fabrics therefrom, and more particularly to propylene-based fibers having low denier, the fabrics having a low basis weight and small pore size.

### BACKGROUND

It would be desirable to produce meltspun fibers having smaller diameter than possible with current polypropylene grades. Meltspun fabrics, for example, those produced by spunbond techniques, comprising finer fibers would have more fibers per unit volume (or area) than would comparable basis weight (mass per unit area) meltspun fabrics produced with larger diameter fibers. The smaller diameter fibers of such meltspun fabrics would provide several advantages. The increased number of fibers per unit area would increase the opacity of the fabric thereby increasing its visual aesthetics. Small diameter fibers also provide a more uniform, consistent layer of fibers with fewer “thin spots” than found in fabrics prepared with larger diameter fibers. The improved uniformity (or “coverage”) provides additional performance advantages to meltspun fabrics and laminates containing these meltspun fabrics. Specific performance enhancements include, but are not limited to, reducing the average pore size of the nonwoven fabrics.

Reducing pore size in meltspun fabrics would enhance containment of solid materials such as superabsorbent polymers that may be present in hygiene articles such as infant diapers, adult incontinence products, or other absorbent products. Reduced fiber diameter, and the attendant increase in the number of fibers per unit volume/area would result in fabrics having higher tensile strength than similar basis weight fabrics prepared of larger diameter fibers. Increased fabric strength may be desirable, for example, to increase the durability of protective clothing, but perhaps of more interest is the ability to reduce fabric basis weight and maintain tensile strength. It is desired that polymers used in the production of meltspun fabrics and laminates thereof exhibit good tensile properties over a broad range of processing conditions, specifically, over a broad range of calender bonding temperatures. It is further desirable that high tensile strength be obtained at as low a calender temperature as possible. Low calendering temperature affords potential energy savings (e.g., reduced heating required). Low calendering temperature also reduces the potential for calendering operations to “burn-through” low basis weight fabrics, creating unacceptable “pin-holes” and/or hard spots in the meltspun fabric or laminate.

The inventors have found that producing meltspun fibers and fabrics comprising such using a polypropylene having a relatively high melt flow rate, narrow molecular weight distribution and having a regio-defect structure such as to reduce the peak average crystalline melting point will solve these and other problems.

Related disclosures include US 2009/0022956, U.S. Pat. No. 6,583,076, U.S. Pat. No. 5,723,217 and U.S. Pat. No. 5,726,103.

### SUMMARY

Disclosed herein are nonwoven fabrics of fibers comprising one or more primary polypropylenes having a molecular weight distribution of less than 3.5 and a melt flow rate within the range from 5 to 500 dg/min, the fibers having an average diameter of less than 20 μm, or a denier (g/9000 m) of less than 2.0, thus forming propylene-based fabrics. The primary polypropylene is preferably a reactor grade polymer made using a single-site catalyst.

In certain embodiments, the propylene-based fabrics disclosed herein have a MD Tensile Strength (WSP 110.4 (05)) of greater than 20 N/5 cm when calendered at a temperature within the range from 110 to 150° C. Also in certain embodiments, the fabrics have a CD Tensile Strength (WSP 110.4 (05)) of greater than 10 N/5 cm when calendered at a temperature within the range from 110 to 150° C. The fabrics are preferably meltspun, and in a particular embodiment are spunbond fabrics.

Also disclosed herein are methods of forming a propylene-based fabric comprising meltspinning one or more primary polypropylenes having a molecular weight distribution of less than 3.5 and a melt flow rate within the range from 5 to 500 dg/min in a meltspun process at fiber forming velocities of greater than 3000 m/min to produce fibers having an average diameter of less than 20 μm, or an denier (g/9000 m) of less than 2.0. In a particular embodiment, the extruded fibers are exposed to an attenuating air pressure of greater than 2000 Pa.

The various descriptive elements and numerical ranges disclosed herein can be combined with other descriptive elements and numerical ranges to describe preferred embodiments of the propylene-based fibers, fabrics and laminates that comprise such; further, any upper numerical limit of an element can be combined with any lower numerical limit of the same element to describe preferred embodiments. In this regard, the phrase “within the range from X to Y” is intended to include within that range the “X” and “Y” values.

### BRIEF DESCRIPTION OF THE DRAWINGS

FIG. 1 is a graphical representation of the fiber diameter of inventive and comparative fibers as a function of attenuating air pressure (“cabin pressure”) used to make the fibers in the exemplary spunbond process.

### DETAILED DESCRIPTION

Certain polypropylenes, called “first polypropylenes” herein, are used to make nonwoven fibers and fabrics (“propylene-based fabrics”) that have improved properties compared to currently used fabrics for such applications as diapers, bandages, etc. These first polypropylenes are made from single-site catalysts such as metallocenes in certain embodiments, and are reactor grade polymers meaning that they have not undergone any post-production process that alters their molecular weight such as by controlled rheology. The first polypropylenes are particularly suited for making fibers and fabrics in a meltspun process such as meltblown, spunbond and/or coform process. The fibers are relatively fine, having an average diameter of less than 20 μm, or a denier (g/9000 m) of less than 2.0, or both. These attributes impart desirable properties to the fabrics made therefrom such as a small average pore volume and the ability to make lighter (lower basis weight) fabrics that maintain their strength. Further, the first polypropylenes are such that they can be used to make fine fibers using relatively high throughputs and high attenuation force or pressure.

As used herein, “Group” refers to the new notation for groups of the Period Table of Elements as in HAWLEY'S CONDENSED CHEMICAL DICTIONARY (R. J. Lewis, Sr. Wiley & Sons, Inc. 1997).

As used herein, “meltspun” refers to a fabric made by a method of forming a web of fibers (“fabric”) in which a polymeric melt or solution is extruded through spinnerets to form filaments which are then attenuated by an appropriate means and laid down on a moving screen, drum or other suitable device. Meltspinning processes include, but are not limited to, spunbonding, flash spinning, coforming, and meltblowing. Meltspun fibers typically have an average diameter of less than 250 or 150 or 60 or 40 μm. Non-limiting examples of suitable polymers used to make meltspun fibers are polypropylene (e.g., homopolymers, copolymers, impact copolymers), polyester (e.g., PET), polyamide, polyurethane (e.g., Lycra™), polyethylene (e.g., LDPE, LLDPE, HDPE, plastomers), polycarbonate, and blends thereof.

As used herein, “spunbond” refers to a meltspinning method of forming a fabric in which a polymeric melt or solution is extruded through spinnerets to form filaments which are cooled then attenuated by suitable means such as by electrostatic charge or high velocity air, such attenuated filaments (“fibers”) then laid down on a moving screen to form the fabric. Fibers resulting from a spunbond process typically have some degree of molecular orientation imparted therein. As used herein, “meltblown” refers to a method of forming a fabric in which a polymeric melt or solution is extruded through spinnerets to form filaments which are attenuated by suitable means such as by electrostatic charge or high velocity air, such attenuated filaments (“fibers”) are then laid down on a moving screen to form the fabric. The fibers themselves may be referred to as being “spunbond” or “meltblown.” Spunbond and meltblown fibers may have any desirable average diameter, and in certain embodiments are within the range from 0.1 or 1 or 4 to 15 or 20 or 40 or 50 or 150 or 250 μm, or expressed another way, a denier (g/9000 m) of less than 2.0 or 1.9 or 1.8 or 1.6 or 1.4 or 1.2 or 1.0.

As used herein, the term “coform” refers to another meltspinning process in which at least one meltspun die head is arranged near a chute through which other materials are added to the fabric while it is forming Such other materials may be pulp, superabsorbent particles, cellulose or staple fibers, for example. Coform processes are shown in U.S. Pat. No. 4,818,464 and U.S. Pat. No. 4,100,324. For purposes of this disclosure, the coform process is considered a particular embodiment of meltspun processes. In certain embodiments, the propylene-based fabrics described herein are coform fabrics.

As used herein, a “fiber” is a structure whose length is very much greater than its diameter or breadth; the average diameter is on the order of 0.1 to 250 μm, and comprises natural and/or synthetic materials. Fibers can be “mono-component” or “bi-component”. Bicomponent fibers comprise two or more polymers of different chemical and/or physical properties extruded from separate extruders but the same spinnerets with both polymers within the same filament, resulting in fibers having distinct domains. The configuration of such a bicomponent fiber may be, for example, sheath/core arrangement wherein one polymer is surrounded by another or may be side-by-side as in U.S. Pat. No. 5,108,820, or “islands in the sea” such as in U.S. Pat. No. 7,413,803. Fibers can also be “mono-constituent” or “bi-constituent”, meaning that they are made of a single polymer or a blend of two or more polymers. In a particular embodiment the propylene-based fibers described herein are mono-component and mono-constituent.

As used herein, a “laminate” comprises at least two fabrics and/or film layers. Laminates may be formed by any means known in the art. Such a laminate may be made for example by sequentially depositing onto a moving forming belt first a meltspun fabric layer, then depositing another meltspun fabric layer or adding a dry-laid fabric on top of the first meltspun fabric layer, then adding a meltspun fabric layer on top of those layers, followed by some bonding of the laminate, such as by thermal point bonding or the inherent tendency of the layers to adhere to one another, hydroentangling, etc. Alternatively, the fabric layers may be made individually, collected in rolls, and combined in a separate bonding step or steps. Multilayer laminates may also have various numbers of layers in many different configurations and may include other materials like films or coform materials, meltblown and spunbond materials, air-laid materials, etc.

As used herein, materials and/or fabrics referred to as being “elastic” or “elastomeric” are those that, upon application of a biasing force, can stretch to an elongated length of at least 110% of its relaxed, original length without rupture or breakage, but upon release of the biasing force the material shows at least 40% or more recovery of its elongation. Suitable elastomeric materials are described further herein. A material, such as a fabric, is “extensible” if upon application of a biasing force the material can stretch to an elongated length of at least 110% of its relaxed, original length without rupture or breakage, but upon release of the biasing force the material shows less than 40% recovery of its elongation. Extensible fabrics often accompany elastomeric fabric or film layers of a laminate and are formed from a material that is extensible (e.g., polyurethanes, styrenic block copolymers, ethylene vinyl acetates, certain polypropylene copolymers, polyethylenes, and blends thereof), or formed by mechanically distorting or twisting a fabric (natural or synthetic).

As used herein, a “film” is a flat unsupported section of a plastic and/or elastomeric material whose thickness is very narrow in relation to its width and length and has a continuous or nearly continuous macroscopic morphology throughout its structure allowing for the passage of air at diffusion-limited rates or lower. The laminates described herein may include one or more film layers and can comprise any material as described herein for the fabrics. In certain embodiments, films are absent from the laminates described herein. Films described herein may contain additives that, upon treatment, promote perforations and allow the passage of air and/or fluids through the film. Additives such as clays, etc. are well known in the art and described particularly in U.S. Pat. No. 6,632,212.

As used herein, “primary polypropylene” refers to a propylene homopolymer, or a copolymer of propylene, or some mixture of propylene homopolymers and copolymers. In certain embodiments, the primary polypropylene described herein is predominately crystalline, thus the primary polypropylene may have a melting point (Tₘ) of less than 165 or 160 or 155 or 150° C. The term “crystalline,” as used herein, characterizes those polymers which possess high degrees of inter- and intra-molecular order. In certain embodiments the primary polypropylene has a heat of fusion (H_{f}) greater than 40 J/g or 60 J/g or 70 J/g, and within the range from 40 or 50 to 70 or 80 or 100 or 140 or 150 J/g in certain embodiments, as determined by DSC analysis. The heat of fusion is dependent on the composition of the primary polypropylene; the thermal energy for the highest order of primary polypropylene is estimated at 189 J/g that is, 100% crystallinity is equal to a heat of fusion of 189 J/g. A polypropylene homopolymer will have a higher heat of fusion than a copolymer or blend of homopolymer and copolymer.

In certain embodiments, the primary polypropylene(s) are isotactic. Isotacticity of the propylene sequences in the primary polypropylenes can be achieved by polymerization with the choice of a desirable catalyst composition. The isotacticity of the primary polypropylenes as measured by ¹³C NMR, and expressed as meso diad content is within the range from 90% (meso diads [m]>0.90) or 95% or 97% to 98% or 99% in certain embodiments, determined as in U.S. Pat. No. 4,950,720 by ¹³C NMR. Expressed another way, the isotacticity of the primary polypropylenes as measured by ¹³C NMR, and expressed as pentad content, is within the range from 60% or 70% to 97% or 98% or 99% in certain embodiments. Alternately described, suitable primary polypropylenes have within the range from 0.1 or 0.5 to 1 mole % to 2 or 3 or 4 or 5 or 8 or 15 mole % regio defects as measured by ¹³C NMR. As used herein, “regio defect” means the insertion of the monomer unit in the opposite direction relative to the prevailing insertion direction. With propylene as an example, with the methylene carbon labeled as “1” and the ethylene carbon labeled as “2”, the mis-insertion would be that of a 2,1 insertion relative to the usual 1,2 insertion.

The primary polypropylene can vary widely in composition. For example, substantially isotactic primary polypropylene homopolymer or propylene copolymer containing equal to or less than 10 wt % of other monomer, that is, at least 90 wt % by weight propylene can be used. Further, the primary polypropylene can be present in the form of a graft or block copolymer, in which the blocks of primary polypropylene have substantially the same stereoregularity as the propylene-α-olefin copolymer so long as the graft or block copolymer has a sharp melting point above 110° C. or 115° C. or 130° C., characteristic of the stereoregular propylene sequences. The primary polypropylene may be a combination of homopolypropylene, and/or random, and/or block copolymers as described herein. When the primary polypropylene is a random copolymer, the percentage of the α-olefin derived units in the copolymer is, in general, up to 5% by weight of the primary polypropylene, 0.5% to 5% by weight in another embodiment, and 1% to 4% by weight in yet another embodiment. The preferred comonomer is derived from ethylene or α-olefins containing 4 to 12 carbon atoms. One, two or more comonomers can be copolymerized with propylene. Exemplary α-olefins may be selected from the group consisting of ethylene; 1-butene; 1-pentene-2-methyl-1-pentene-3-methyl-1-butene; 1-hexene-3-methyl-1-pentene-4-methyl-1-pentene-3,3-dimethyl-1-butene; 1-heptene; 1-hexene; 1-methyl-1-hexene; dimethyl-1-pentene; trimethyl-1-butene; ethyl-1-pentene; 1-octene; methyl-1-pentene; dimethyl-1-hexene; trimethyl-1-pentene; ethyl-1-hexene; 1-methylethyl-1-pentene; 1-diethyl-1-butene; propyl-1-pentene; 1-dec ene; methyl-1-nonene; 1-nonene; dimethyl-1-octene; trimethyl-1-heptene; ethyl-1-octene; methylethyl-1-butene; diethyl-1-hexene; 1-dodecene and 1-hexadodecene.

The molecular weight of the primary polypropylene is adjusted in situ while being produced in the reactor (“reactor grade”) by techniques well known in the art such by the addition to the reactor of a chain-terminating agent, an example of which is hydrogen. The primary polypropylene is preferably a reactor grade polypropylene. The weight average molecular weight (M_{w}) of the primary polypropylene is within the range from 50,000 to 800,000 g/mol in one embodiment, or from 60,000 to 600,000 g/mol in another embodiment. The primary polypropylene possesses a number average molecular weight (Mₙ) value within the range from 25,000 to 60,000 in one embodiment, and from 30,000 to 100,000 in yet another embodiment. The molecular weight distribution (MWD, Mw/Mn) of the primary polypropylene is within the range from 1.5 to 2.5 or 3.0 or 4.0 or 5.0 in certain embodiments, and is less than 3.5 or 3.0 or 2.5 in yet other embodiments. The primary polypropylene possesses a z-average molecular weight (M_{z}) value of from 200,000 to 600,000 in one embodiment, and from 300,000 to 550,000 in yet another embodiment, and from greater than 200,000 or 300,000 or 400,000 or 500,000 in certain embodiments. In other embodiments, the primary polypropylene possesses a M_{z}/M_{w} of from greater than 2.0, and greater than 2.1 in another embodiment, and from greater than 2.2 in yet another embodiment, and in yet other embodiments the M_{z}/M_{w} is within the range from 2.0 or 2.1 or 2.2 or 2.3 to 2.8 or 3.0 or 3.5 or 3.8 or 4.0 or 4.5 or 5.0 or 6.0 or 7.0. The primary polypropylene has an MFR (2.16 kg/230° C.) of less than 100 or 80 or 70 or 60 or 55 dg/min in certain embodiments; and the MFR is within the range from 5 or 10 or 20 or 30 to 100 or 150 or 200 or 300 or 500 dg/min in other embodiments.

There is no particular limitation on the method for preparing the primary polypropylenes described herein. For example, the polypropylene may be formed by Ziegler-Natta catalysis, or preferably formed by single-site catalysis. Suitable single-site catalysts include, but are not limited to, Group 4-10 metallocenes, Group 4-10 constrained geometry catalysts, and Group 4-10 amine or diimine-coordination compounds with, each with suitable activators. In a particular embodiment, a Group 4 metallocene is used in conjunction with the appropriate activator to catalyze the primary polypropylene. Metallocenes are described throughout in, for example, 1 & 2 METALLOCENE-BASED POLYOLEFINS (John Scheirs & W. Kaminsky eds., John Wiley & Sons, Ltd. 2000). In any case, the primary polypropylene may be obtained by homopolymerization of propylene in a single stage or multiple stage reactor. Copolymers may be obtained by copolymerizing propylene and ethylene or an α-olefin having from 4 to 20 carbon atoms in a single stage or multiple stage reactor. Polymerization methods include, but are not limited to, high pressure, slurry, gas, bulk, or solution phase, or a combination thereof.

Exemplary commercial primary polypropylenes include the family of Achieve™ polymers (ExxonMobil Chemical Company, Baytown, Tex.). The Achieve polymers are produced based on metallocene catalyst system. In certain embodiments, the metallocene catalyst system produces a narrow molecular weight distribution polymer. The MWD is within the range from 1.5 to 3.0 in certain embodiments, and from 1.5 to 2.5 in another embodiment. However, a broader MWD polymer may be produced in a process with multiple reactors. Different MW polymers can be produced in each reactor to broaden the MWD. Other primary polypropylene random copolymer and impact copolymer may also be used.

Although the “primary polypropylene” component of the fiber and fabric compositions is sometimes discussed as a single polymer, also contemplated by the term are blends of two or more different polypropylenes which, when combined result in a polymer composition having the properties within the ranges described herein. In certain embodiments, the primary polypropylene may be present in the fabric within the range from 75 or 70 to 80 or 90 or 95 or 99 or 99.9 wt %, by weight of the fabric layer/composition. The blend may include, but is not limited to, other polypropylenes (impact copolymers, random copolymers, elastomeric polypropylenes), polyester, polyamide, polyurethane, polyethylene, an elastomer (as described herein), and blends thereof. These and other suitable materials are well known in the art and elucidated further herein. Fibers made with such blends are called “biconstituent” fibers, and are not limited to having only two different polymers blended together. Further, such blends are not limited by the level of miscibility of the polymers and may in fact form bi-phasic blends in certain embodiments.

Particularly disclosed herein are nonwoven fabrics produced using primary polypropylenes (“propylene-based fabrics”) or compositions including such primary polypropylenes. The nonwoven fabrics are meltspun fabrics in certain embodiments, and are spunbond in a particular embodiment. The spunbonding process in certain embodiments involves the process of melt-extruding the desired material through one or more spinnerets comprising at least one die having small diameter holes, the stream of molten material then being attenuated (drawn) by pressurized air, creating a venturi effect. The material may be added to the melt-extruder as pellets having desirable additives, or additives may be combined in this step.

In particular, the formation of primary polypropylene filaments is accomplished by extruding the molten material through an appropriate die comprising a plurality of spinnerets (capillaries, holes) as known in the art, followed by quenching the molten material (having a desirable melt temperature within the die) with a quench air system the temperature of which may be controlled. Common quench air systems include those that deliver temperature controlled air in a cross-flow direction. Filaments are then pulled away from the one or more spinnerets and thus attenuated. To accomplish this, the filaments are attenuated by passing through a venturi device in which due to pressurized air flow, accelerates and/or attenuates the filaments. Increasing the increasing the air velocity within the venturi device may be done by a variety of methods described in the art, including raising the air pressure within the venturi device. Typically, increasing this air velocity (for example by increasing air pressure) results in increased filament velocity and greater filament attenuation. The higher the air pressure, the more the primary polypropylene is accelerated and so attenuated, in terms of speed and denier of the fiber that is formed therefrom. To achieve finer fibers, high air pressures are desirable. However, this must be balanced by the tendency for the filaments to break due to excessive pressure. The primary polypropylenes described herein can be attenuated using higher air pressures than is typical in other spunbond processes. In one embodiment, the attenuating air pressure used in the spunbonding process is greater than 2000 or 3000 or 4000 or 6000 Pa, and less than 600 or 500 or 400 kPa in other embodiments; and is within the range from 2000 or 3000 or 4000 to 8000 or 10,000 or 15,000 Pa in other embodiments. Such air pressure may be generated in a closed area where the fibers are attenuated such as a “cabin”, and the air pressure therein is sometimes referred to as a “cabin pressure.”

It is well known in the art how air attenuation can be accomplished and the process is not limited to any particular method of attenuating the filaments. In one embodiment, the venturi effect is obtained by drawing the filaments of primary polypropylene using an aspirator slot (slot draw), which runs the width of the machine. In another embodiment, the venturi effect is obtained by drawing the filaments through a nozzle or aspirator gun. Multiple guns can be used, since orifice size can be varied to achieve the desired effect. Filaments of the primary polypropylene thus formed are collected onto a screen (“wire”) in one embodiment, or porous forming belt in another embodiment to form a fabric of the filaments. Typically, a vacuum is maintained on the underside of the belt to promote the formation of a uniform fabric and to remove the air used to attenuate the filaments and creating the air pressure. The actual method of air attenuation is not critical, as long as the desirable accelerating air velocity, (often reflected by the air pressure), and hence venturi effect, is obtained to attenuate the primary polypropylene filaments.

Pressure in the die block in one embodiment is generated by a gear pump. The method of forming the pressure in the die block is not critical, but the pressure inside the die block ranges from 35 to 50 bar (3500 to 5000 kPa) in one embodiment, and from 36 to 48 bar (3600 to 4800 kPa) in another embodiment, and from 37 to 46 bar (3700 to 4600 kPa) in yet another embodiment.

The melt temperature in the die of the primary polypropylene melt ranges from 200 to 260° C. in one embodiment, and from 200 to 250° C. in yet another embodiment, and ranges from 210 to 245° C. in yet another embodiment.

Any number of spinnerets including any number of dies can be used. In one embodiment, a die is used that contains from 4000 to 9000 holes per meter, and from 4500 to 8500 holes per meter in another embodiment, and from 5000 to 8000 holes per meter in yet another embodiment, wherein any upper die hole limit may be combined with any lower die hole to obtain a desirable range of die holes.

In certain embodiments, the spunbond line throughput is within the range from 150 or 170 to 200 or 270 to 300 kg/hr. In certain other embodiments, the spunbond line throughput per hole is within the range from 0.20 or 0.30 or 0.40 to 0.60 or 0.70 or 0.90 ghm.

In forming propylene-based fabrics, there are any number of ways of dispersing or distributing the filaments to form a uniform fabric. In one embodiment, a deflector is used, either stationary or moving. In another embodiment, static electricity or air turbulence is used to improve fabric uniformity. Other means may also be used as is known in the art. In any case, the formed fabric typically passes through compression rolls to improve fabric integrity. The fabric, in one embodiment, is then passed between heated calender rolls where the raised lands on one roll bond the fabric at certain points to further increase the spunbonded fabric integrity. The compression and heated calender can be isolated from the area where the filaments are formed in one embodiment.

Various additional potential processing and/or finishing steps known in the art, such as slitting, treating, printing graphics, etc., may be performed without departing from the spirit and scope of the invention. For instance, the fabric or laminate comprising the fabric may optionally be mechanically stretched in the cross-machine and/or machine directions to enhance extensibility. In one embodiment, the fabric or laminate may be coursed through two or more rolls that have grooves in the CD and/or MD directions. Such grooved satellite/anvil roll arrangements are described in US 2004/0110442 and US 2006/0151914 and U.S. Pat. No. 5,914,084. For instance, the fabric or laminate may be coursed through two or more rolls that have grooves in the CD and/or MD directions. The grooved rolls may be constructed of steel or other hard material (such as a hard rubber). If desired, heat may be applied by any suitable method known in the art, such as heated air, infrared heaters, heated nipped rolls, or partial wrapping of the fabric or laminate around one or more heated rolls or steam canisters, etc. Heat may also be applied to the grooved rolls themselves. It should also be understood that other grooved roll arrangement are equally suitable, such as two grooved rolls positioned immediately adjacent to one another. Besides grooved rolls, other techniques may also be used to mechanically stretch the composite in one or more directions. For example, the composite may be passed through a tenter frame that stretches the composite. Such tenter frames are well known in the art and described, for instance, in US 2004/0121687.

No matter how formed and calendered, the propylene-based fabrics comprise fibers having an average diameter of less than 20 or 17 or 15 or 12 μm in certain embodiments, a denier (g/9000 m) of less than 2.0 or 1.9 or 1.8 or 1.6 or 1.4 or 1.2 or 1.0 in certain embodiments, or both. Such fabrics, when calendered at a temperature (calender set temperature) within the range from 110 to 150° C. have a MD Tensile Strength (WSP 110.4 (05)) of greater than 20 or 25 N/5 cm in certain embodiments. The fabrics have a CD Tensile Strength (WSP 110.4 (05)) of greater than 10 or 15 N/5 cm when calendered at a temperature (calender set temperature) within the range from 110 to 150° C. in other embodiments.

In certain embodiments, the propylene-based fabrics have an average pore size within the range of from 10 or 25 or 50 to 100 or 200 μm as determined from photomicrograph studies. In yet other embodiments, the fabrics have a basis weight of from less than 14 or 13 or 12 or 11 g/m², and in other embodiments, within the range from 0.1 or 1 or 2 to 11 or 14 g/m².

In certain embodiments, the fibers used to form the propylene-based fabrics are bicomponent or “conjugate” fibers. These include structures that are side-by-side, segmented, sheath/core, island-in-the-sea structures (“matrix fibril”), and others as is known in the art. In these structures, at least one of the polymers used to make the fiber is the primary polypropylene. The second, third, etc. component of the conjugate fiber may be made from any suitable materials such as polypropylene, polyethylene (e.g., LDPE, LLDPE, HDPE), plastomers (ethylene-α-olefin copolymers), polyurethane, polyesters such as polyethylene terephthanlate, polylactic acid, polyvinyl chloride, polytetrafluoroethylene, styrenic block copolymers, propylene-α-olefin elastomers (e.g., Vistmaxx) ethylene vinyl acetate copolymers, polyamide, polycarbonate, cellulosics (e.g., cotton, Rayon™, Lyocell™, Tencil™), wood, viscose, and blends of any two or more of these materials. A particularly preferred second (or third, etc.) component is a polyethylene. The main objective of producing bicomponent fibers is to exploit capabilities not existing in either polymer alone. By this technique, it is possible to produce fibers of any cross sectional shape or geometry that can be imagined. Side-by-side fibers are generally used as self-crimping fibers. There are several systems used to obtain a self-crimping fiber. One of them is based on different shrinkage characteristics of each component. There have been attempts to produce self-crimping fibers based on different electrometric properties of the components. Some types of side-by-side fibers crimp spontaneously as the drawing tension is removed and others have “latent crimp”, appearing when certain ambient conditions are obtained. In some embodiments “reversible” and “non-reversible” crimps are used, when reversible crimp can be eliminated as the fiber is immersed in water and reappears when the fiber is dried. This phenomenon is based on swelling characteristics of the components. Different melting points on the sides of the fiber are taken advantage of when fibers are used as bonding fibers in thermally bonded non-woven webs. Sheath-core bicomponent fibers are those fibers where one of the components (core) is fully surrounded by the second component (sheath). Adhesion is not always essential for fiber integrity. The most common way of production of sheath-core fibers is a technique where two polymer liquids are separately led to a position very close to the spinneret orifices and then extruded in sheath-core form. In the case of concentric fibers, the orifice supplying the “core” polymer is in the center of the spinning orifice outlet and flow conditions of core polymer fluid are strictly controlled to maintain the concentricity of both components when spinning. Eccentric fiber production is based on several approaches: eccentric positioning of the inner polymer channel and controlling of the supply rates of the two component polymers; introducing a varying element near the supply of the sheath component melt; introducing a stream of single component merging with concentric sheath-core component just before emerging from the orifice; and deformation of spun concentric fiber by passing it over a hot edge. Matrix fibril fibers are spun from the mixture of two polymers in the required proportion; where one polymer is suspended in droplet form in the second melt. An important feature in production of matrix-fibril fibers is the necessity for artificial cooling of the fiber immediately below the spinneret orifices. Different spinnability of the two components would almost disable the spinnability of the mixture, except for low concentration mixtures (less than 20%). Bicomponent fibers are used to make fabrics that go into such products as diapers, feminine care and adult incontinence products (as top sheet, back sheet, leg cuffs, elastic waistband, transfer layers); air-laid nonwoven structures are used as absorbent cores in wet wipes; and used in spun laced nonwoven products like medical disposable textiles, filtration products.

Also in certain embodiments the propylene-based fabric may be a mixed-fiber fabric comprising propylene-based fibers. Mixed-fiber fabrics are disclosed in, for example, US 2008/0038982. There can be one, two or more other types of fibers with the propylene-based fibers include fibers made from polypropylene, polyethylene, plastomers, polyurethane, polyesters such as polyethylene terephthalate, polylactic acid, polyvinyl chloride, polytetrafluoroethylene, styrenic block copolymers, propylene-α-olefin elastomers (e.g., Vistamaxx™) or other elastomers as described herein, ethylene vinyl acetate copolymers, polyamide, polycarbonate, cellulosics (e.g., cotton, Rayon™, Lyocell™, Tencil™), wood, viscose, and blends of any two or more of these materials.

In certain embodiments, the one or more propylene-based fabrics may form a laminate either with itself or with other secondary layers. The lamination of the various layers can be done such that CD and/or MD orientation is imparted into the fabric or laminate, especially in the case where the laminate includes at least one elastomeric layer. Many approaches may be taken to form a laminate comprising an elastomeric film and/or fabric layer which remains elastomeric once the laminate layers are bonded together. One approach is to fold, corrugate, crepe, or otherwise gather the fabric layer prior to bonding it to the elastomeric film. The gathered fabric is bonded to the film at specified points or lines, not continually across the surface of the film. While the film/fabric is in a relaxed state, the fabric remains corrugated or puckered on the film; once the elastomeric film is stretched, the fabric layer flattens out until the puckered material is essentially flat, at which point the elastomer stretching ceases.

Another approach is to stretch the elastomeric film/fabric, then bond the fabric to the film while the film is stretched. Again, the fabric is bonded to the film at specified points or lines rather than continually across the surface of the film. When the stretched film is allowed to relax, the fabric corrugates or puckers over the unstretched elastomeric film.

Another approach is to “neck” the fabric prior to bonding it to the elastomer layer as described in U.S. Pat. No. 5,336,545, U.S. Pat. No. 5,226,992, U.S. Pat. No. 4,981,747 and U.S. Pat. No. 4,965,122. Necking is a process by which the fabric is pulled in one direction, which causes the fibers in the fabric to slide closer together, and the width of the fabric in the direction perpendicular to the pulling direction is reduced. If the necked fabric is point-bonded to an elastomeric layer, the resulting laminate will stretch somewhat in a direction perpendicular to the direction in which the fabric was pulled during the necking process, because the fibers of the necked fabric can slide away from one another as the laminate stretches.

Yet another approach is to activate the elastomeric laminate once it has been formed. Activation is a process by which the elastomeric laminate is rendered easy to stretch. Most often, activation is a physical treatment, modification or deformation of the elastomeric laminate, said activation being performed by mechanical means. For example, the elastomeric laminate may be incrementally stretched by using intermeshing rollers, as discussed in U.S. Pat. No. 5,422,172, or US 2007/0197117 to render the laminate stretchable and recoverable. Finally, the elastomeric film or fabric may be such that it needs no activation and is simply formed onto and/or bound to a secondary layer to form an elastic laminate.

Such processes can also be used on non-elastomeric laminates to improve other properties such as drape and softness.

In any case, the laminates described herein comprise one or more secondary layers, the secondary layers comprising other fabrics, nets, coform fabrics, scrims, and/or films, any of which are prepared from natural materials, synthetic materials, or blends thereof. The materials may be extensible, elastic or plastic in certain embodiments. In particular embodiments, the one or more secondary layers comprise materials selected from the group consisting of polypropylene, polyethylene, plastomers, polyurethane, polyesters such as polyethylene terephthanlate, polylactic acid, polyvinyl chloride, polytetrafluoroethylene, styrenic block copolymers, ethylene vinyl acetate copolymers, polyamide, polycarbonate, cellulosics (e.g., cotton, Rayon™, Lyocell™, Tencil™), wood, viscose, and blends of any two or more of these materials. Any secondary layer may also comprise (or consist essentially of) any material that is elastic, examples of which include propylene-α-olefin elastomer (e.g., Vistamaxx™ propylene-based elastomer sold by ExxonMobil Chemical), natural rubber (NR), synthetic polyisoprene (IR), butyl rubber (copolymer of isobutylene and isoprene, IIR), halogenated butyl rubbers (chloro-butyl rubber: CIIR; bromo-butyl rubber: BIIR), polybutadiene (BR), styrene-butadiene rubber (SBR), nitrile rubber, hydrogenated nitrile rubbers, chloroprene rubber (CR), polychloroprene, neoprene, EPM (ethylene-propylene rubber) and EPDM rubbers (ethylene-propylene-diene rubber), epichlorohydrin rubber (ECO), polyacrylic rubber (ACM, ABR), silicone rubber, fluorosilicone rubber, fluoroelastomers, perfluoroelastomers, polyether block amides (PEBA), chlorosulfonated polyethylene (CSM), ethylene-vinyl acetate (EVA), thermoplastic elastomers (TPE), thermoplastic vulcanizates (TPV), thermoplastic polyurethane (TPU), thermoplastic olefins (TPO), polysulfide rubber, or blends of any two or more of these elastomers. In certain embodiments, the one or more elastic layers comprise propylene-α-olefin elastomer, styrene-butadiene rubber, or blends thereof. In yet other embodiments, the one or more elastic layers consist essentially of propylene-α-olefin elastomer(s). In a particular embodiment, styrenic-based elastomers (polymers comprising at least 10 wt % styrene or substituted-styrene-derived units) are absent from the multilayer fabric.

The secondary layer(s) may be in the form of films, fabrics, or both. Films may be cast, blown, or made by any other suitable means. When the secondary layers are fabrics, the secondary layers can be meltspun, dry-laid or wet-laid fabrics. The dry-laid processes include mechanical means, such as how carded fabrics are produced, and aerodynamic means, such as, air-laid methods. Dry-laid nonwovens are made with staple fiber processing machinery such as cards and garnetts, which are designed to manipulate staple fibers in the dry state. Also included in this category are nonwovens made from fibers in the form of tow, and fabrics composed of staple fibers and stitching filaments or yarns, namely, stitchbonded nonwovens. Fabrics made by wet-laid processes made with machinery associated with pulp fiberizing, such as hammer mills, and paperforming. Web-bonding processes can be described as being chemical processes or physical processes. In any case, dry- and wet-laid fabrics can be jet and/or hydroentangled to form a spunlace fabric as is known in the art. Chemical bonding refers to the use of water-based and solvent-based polymers to bind together the fibrous webs. These binders can be applied by saturation (impregnation), spraying, printing, or application as a foam. Physical bonding processes include thermal processes such as calendering and hot air bonding, and mechanical processes such as needling and hydroentangling. Spunlaid nonwovens are made in one continuous process: fibers are spun by melt extrusion and then directly dispersed into a web by deflectors or can be directed with air streams.

More particularly, “carding” is the process of disentangling, cleaning, and intermixing fibers to make a web for further processing into a nonwoven fabric and is well known in the art. The fabric is called a “carded” fabric when made using this process. The aim is to take a mass of fiber tufts and produce a uniform, clean web. An example of a method of carding is described in U.S. Pat. No. 4,105,381. The process predominantly aligns the fibers which are held together as a web by mechanical entanglement and fiber-fiber friction. The main type of card is a roller card. The carding action is the combing or working of fibers between the points of saw-tooth wire clothing on a series of interworking card rollers. Short fibers and foreign bodies are removed, the fiber tufts are opened, and the fibers are arranged more or less parallel. The carding or parallelization of fibers occurs when one of the surfaces moves at a speed greater than the other. Fibers are removed, or “stripped,” when the points are arranged in the same direction and the more quickly moving surface removes or transfers the fibers from the more slowly moving surface.

High speed cards designed to produce nonwoven webs may be configured with one or more main cylinders, roller or stationary tops, one or two doffers, or various combinations of these principal components. Single-cylinder cards are usually used for products requiring machine-direction or parallel-fiber orientation. Double-cylinder cards (or “tandem” cards) are basically two single-cylinder cards linked together by a section of stripper and feed rolls to transport and feed the web from the first working area to the second. The coupling of two carding units in tandem distributes the working area and permits greater fiber throughput at web quality levels comparable to slower single-cylinder machines. Roller-top cards have five to seven sets of workers and strippers to mix and card the fibers carried on the cylinder. The multiple transferring action and re-introduction of new groupings of fibers to the carding zones provides a doubling effect which enhances web uniformity. Stationary-top cards have strips of metallic clothing mounted on plates positioned concavely around the upper periphery of the cylinder. The additional carding surfaces thus established provide expanded fiber alignment with minimum fiber extraction.

In certain embodiments, the propylene-based polymer may be formed into coform fabrics. Methods for forming such fabrics are described in, for example, U.S. Pat. No. 4,818,464 and U.S. Pat. No. 5,720,832. Generally, fabrics of two or more different thermoplastic and/or elastomeric materials may be formed. For example, the coform fabrics described herein may comprise from 1 or 5 or 10 or 20 or 40 or 50 to 60 or 70 or 80 or 90 or 99 wt % of the primary polypropylene and from 99 or 90 or 80 or 70 or 60 to 50 or 40 or 20 or 10 or 5 or 1 wt % of another thermoplastic material such as another polypropylene, polyethylene, polyurethane, etc., or an elastomer such as a propylene-based elastomer or a styrenic block copolymer. Thus, in one aspect is provided the introduction of molten extruded primary polypropylene and optionally one or more other materials (elastomeric, adsorbent, thermoplastic, etc.) to the shear layers of at least one rapidly moving stream or jet of an inert gas from two or more extrusion openings or sets of openings placed surrounding or on alternate or opposite sides of the high velocity gas delivery nozzle. The thermoplastic material which is extruded from these openings may be the same material or, alternatively, materials which differ from one another in their chemical and/or physical properties. Designated as first, second, etc., thermoplastic, absorbent or elastomeric material, the materials may be of the same or different chemical composition or molecular structure and, when of the same molecular structure, may differ in molecular weight or other characteristics which results in differing physical properties. In those situations in which thermoplastic materials are used which differ from one another in some respect, such as in physical properties, the extrusion or die head will be provided with multiple chambers, one for each of the thermoplastic materials, such as first, second, etc., thermoplastic materials. That is, the die head is provided with a first chamber for the first thermoplastic material and a second chamber for the second thermoplastic material, etc. In contrast, such an arrangement where a single chamber is provided with conduits or passages which provide communication between the single chamber and each of the first and the second thermoplastic extrusion outlet openings, when a first chamber and a second chamber are employed for first and second thermoplastic materials, respectively, each chamber is provided with passages to only one extrusion outlet opening or set of openings. Thus, the first thermoplastic material chamber communicates with the first extrusion outlet opening by means of the first thermoplastic material passage, while the second thermoplastic material chamber communicates with the second thermoplastic extrusion opening through the second thermoplastic material passage.

The two or more various layers of fabrics or fibers that make up the laminates described herein may be bound in some manner. As used herein, “bound” (or “bond” or “adhered”) means that two or more fabrics, or a plurality of fibers, is secured to one another through i) the inherent tendency of the molten or non-molten materials' ability to adhere through chemical interactions and/or ii) the ability of the molten or non-molten fibers and/or fabric to entangle with the fibers comprising another material to generate a linkage between the fibers or fabrics. The layers of the laminates described herein may be laminated (bonded) to one another by known methods including heat bonding methods such as hot embossing, spot bonding, calendering, and ultrasonic bonding; mechanical entangling methods such as needle pouncing and water jetting; use of adhesives such as hot melt adhesives and urethane adhesives; and extrusion lamination. Adhesives may be used to facilitate bonding of fabric layers, but in a particular embodiment, adhesives are absent from the fabric layers (not used to bond the fibers of a fabric) described herein; and in another embodiment, absent from the laminates (not used to bond adjacent fabric layers) described herein. Examples of adhesives include those comprising low weight average molecular weight (<80,000 g/mole) polyolefins, polyvinyl acetate polyamide, hydrocarbon resins, natural asphalts, styrenic rubbers, and blends thereof.

The propylene-based fabric or any other film and/or fabric layer of the laminates described herein may include other additives. The additives may be present at any desirable level, examples of which include from 0.1 to 3 or 4 or 5 or 10 wt %, by weight of the fiber or fabric. As used herein, “additives” include, for example, stabilizers, surfactants, antioxidants, anti-ozonants (e.g., thioureas), fillers, migrating (preventative) agent, colorants, nucleating agents, anti-block agents, UV-blockers/absorbers, hydrocarbon resins (e.g., Oppera™ resins, Picolyte™ tackifiers, polyisobutylenes, etc.) and other tackifiers, oils (e.g., paraffinic, mineral, aromatic, synthetic), slip additives, and combinations thereof. Primary and secondary antioxidants include, for example, hindered phenols, hindered amines, and phosphates. Slip agents include, for example, oleamide and erucamide. Examples of fillers include carbon black, clay, talc, calcium carbonate, mica, silica, silicate, and combinations thereof. Other additives include dispersing agents and catalyst deactivators such as calcium stearate, hydrotalcite, and calcium oxide, and/or other acid neutralizers known in the art.

In certain embodiments, the laminates described herein—comprising the one or more propylene-based fabrics characterized by the designation “P”—are selected from structures exemplified by MP, MPM, PP, PPP, PPPP, PPM, PMP, PMMP, PPMPP, PMMPP, PMPPP, PPMMPP, PMPMP, PPPMPP, SP, SPS, PP, PPP, PPPP, DPPPP, MPPPP, SPPPP, PPS, PSP, PSSP, PPSPP, PSSPP, PSPPP, PPSSPP, PSPSP, PPPSPP, DP, DDP, DPD, DPP, DDDDP, PPD, PDP, PDDP, PPDPP, PDDPP, PPDDPP, DMP, DDMPP, PDMDP, DPMPD, DDPMPD, DDPMPDD, DDPMMPDD, DPMMPD, PDMDMD, PMDMP, PDMMDD, PPDMDPP, DDDDMP, PPDMMDPP, FP, MPF, FPP, FPPP, FPPPP, FPPF, FPFPF, FPPM, PFP, PMFP, PPFPP, PFFPP, PMFPP, PPMFPP, PFD, SPFD, SPMD, PDFD, PDDFFD, PDFDD, DPF, DFP, DDDFP, FDP, PDDF, PFDPP, FPDDPP, PFDDPP, DMPF, DFMPP, PDFDP, DPFPD, DDPFPD, DDPFPDD, DDPFFPDD, DPFFPD, PFDFD, PFDFP, SFMP, SSFMP, PFMFP and PFFP, wherein “M” represents meltblown fabric layers, “S” represents spunbond fabric layers, “F” represents film layers, and “D” represents dry-laid (carded or air-laid) fabric layers, each layer being adjacent to one another. In a preferred embodiment, the propylene-based fabric is meltspun, and is spunbond in a particular embodiment. The fabric and/or laminate may be used to form an absorbent or barrier product such as, but not limited to, personal care products, baby diapers, training pants, absorbent underpads, swim wear, wipes, feminine hygiene products, bandages, wound care products, medical garments, surgical gowns, filters, adult incontinence products, surgical drapes, coverings, garments, and cleaning articles and apparatus.

In one embodiment the absorbent article is a disposable diaper as disclosed in, for example, US 2008/0119102 A1 which generally defines a front waist section, a rear waist section, and an intermediate section that interconnects the front and rear waist sections. The front and rear waist sections include the general portions of the diaper which are constructed to extend substantially over the wearer's front and rear abdominal regions, respectively, during use. The intermediate section of the diaper includes the general portion of the diaper that is constructed to extend through the wearer's crotch region between the legs. Thus, the intermediate section is an area where repeated liquid surges typically occur in the diaper. Any one or more of these structures, for example, may comprise the propylene-based fabrics and laminates described herein.

The diaper includes, without limitation, an outer cover, or backsheet, a liquid permeable bodyside liner, or topsheet, positioned in facing relation with the backsheet, and an absorbent core body, or liquid retention structure, such as an absorbent pad, which is located between the backsheet and the topsheet. Any one or more of these structures, for example, may comprise the propylene-based fabrics and laminates described herein. The backsheet defines a length, or longitudinal direction, and a width or lateral direction, which coincide with the length and width of the diaper. The liquid retention structure generally has a length and width that are less than the length and width of the backsheet, respectively. Thus, marginal portions of the diaper, such as marginal sections of the backsheet may extend past the terminal edges of the liquid retention structure. In certain embodiments, the backsheet extends outwardly beyond the terminal marginal edges of the liquid retention structure to form side margins and end margins of the diaper. The topsheet is generally coextensive with the backsheet but may optionally cover an area that is larger or smaller than the area of the backsheet, as desired.

To provide an improved fit and to help reduce leakage of body exudates from the diaper, the diaper side margins and end margins may be elasticized with suitable elastic members. For example, the diaper may include leg elastics constructed to operably tension the side margins of the diaper to provide elasticized leg bands which can closely fit around the legs of the wearer to reduce leakage and provide improved comfort and appearance. Waist elastics are employed to elasticize the end margins of the diaper to provide elasticized waistbands. The waist elastics are configured to provide a resilient, comfortably close fit around the waist of the wearer. The latently elastic materials, such as Vistamaxx™ elastomers which may form a laminate with the propylene-based fabrics described herein are suitable for use as the leg elastics and waist elastics. Exemplary of such materials are laminate sheets that either comprise or are adhered to the backsheet, such that elastic constrictive forces are imparted to the backsheet.

As is known, fastening means, such as hook and loop fasteners, may be employed to secure the diaper on a wearer. Alternatively, other fastening means, such as buttons, pins, snaps, adhesive tape fasteners, cohesives, fabric-and-loop fasteners, or the like, may be employed. In the illustrated embodiment, the diaper includes a pair of side panels (or ears) to which the fasteners, indicated as the hook portion of a hook and loop fastener, are attached. Generally, the side panels are attached to the side edges of the diaper in one of the waist sections and extend laterally outward therefrom. The side panels may be elasticized or otherwise rendered elastomeric by use of latently elastic materials.

The diaper may also include a surge management layer located between the topsheet and the liquid retention structure to rapidly accept fluid exudates and distribute the fluid exudates to the liquid retention structure within the diaper. The diaper may further include a ventilation layer, also called a spacer, or spacer layer, located between the liquid retention structure and the backsheet to insulate the backsheet from the liquid retention structure to reduce the dampness of the garment at the exterior surface of a breathable outer cover, or backsheet. Any one of these structures may comprise the propylene-based fabrics and laminates described herein.

The disposable diaper may also include a pair of containment flaps which are configured to provide a barrier to the lateral flow of body exudates. The containment flaps may be located along the laterally opposed side edges of the diaper adjacent the side edges of the liquid retention structure. Each containment flap typically defines an unattached edge that is configured to maintain an upright, perpendicular configuration in at least the intermediate section of the diaper to form a seal against the wearer's body. The containment flaps may extend longitudinally along the entire length of the liquid retention structure or may only extend partially along the length of the liquid retention structure. When the containment flaps are shorter in length than the liquid retention structure, the containment flaps can be selectively positioned anywhere along the side edges of the diaper in the intermediate section. Such containment flaps are generally well known to those skilled in the art.

### Examples

Three inventive samples and three comparative examples (one metallocene grade hPP and two Ziegler-Natta grade hPPs) were spunbond on a Reicofil 4 line. The three inventive “concept” polypropylene homopolymers were prepared by peroxide cracking of a nominal 22 melt flow rate metallocene-catalyzed propylene polymer granule (base granules for ExxonMobil Achieve™ 3854) to nominally 40, 55, and 70 (all ±5 dg/min) dg/min melt flow rates (“MFR,” ASTM D1238, 2.16 kg, 230° C.). Because the Achieve polypropylene granules as-produced (“reactor grade”) have a narrow molecular weight distribution (Mw/Mn is near the theoretical minimum of 2) it is expected that peroxide cracking will reduce average molecular weight but will not substantially alter (reduce) molecular weight distribution (Mw/Mn) and thus a model for low molecular weight reactor grade metallocene-catalyzed polymers. These concept polymers combine narrow molecular weight distribution with lower than typical molecular weights. The regio-defect structure present in the metallocene-catalyzed polymers contributes to a reduced peak average crystalline melting point, and would contribute to reducing temperatures needed for calender bonding spunbond nonwovens. The combination of narrow molecular weight distribution, low molecular weight, and mPP-specific regio defects provide a balance of rheological properties and melting/crystallization performance.

The samples are described as follows:
Inventive Example 1 is nominal MFR of 40 dg/min, Mw/Mn less than 3.0;
Inventive Example 2 is nominal MFR of 55 dg/min, Mw/Mn less than 3.0;
Inventive Example 3 is nominal MFR of 70 dg/min, Mw/Mn less than 3.0;
Comparative metallocene catalyzed Achieve™ 3854 MFR of 24 dg/min, Mw/Mn less than 3.0;
Comparative Ziegler-Natta catalyzed ExxonMobil PP 3155 MFR of 36 dg/min, Mw/Mn less than 4.0; and
Comparative Ziegler-Natta catalyzed ExxonMobil PP 3885E1 MFR of 65 dg/min.

The inventive “concept” polypropylenes also contain 0.06 wt % Irganox™ 3114, 0.02 wt % calcium stearate, 0.06 wt % Irgafos™ 168, and 0.04 wt % DHT4V. The polymer delivery was fixed at 0.47 gram per hole per minute.

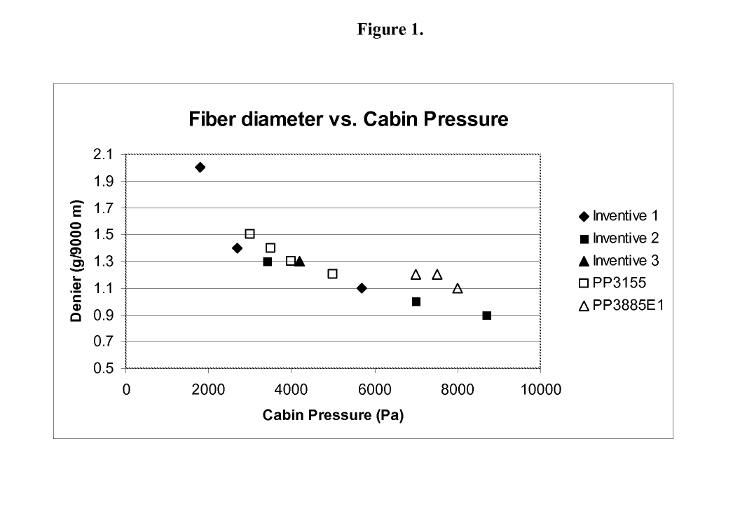

Certain conditions in the Reicofil line were maintained as in the Tables 1-6. The cooling air temperature was fixed at 20° C., and the SAS gap (attenuating unit) was between 20 and 23. In certain experiments, the cabin pressure (or generally, “air pressure”) was increased stepwise and fibers collected for diameter measurements. The highest value tabulated (and graphed) of cabin pressure represents the maximum value at which filament continuity could be maintained. Attempts to run at higher cabin pressures resulted in broken filaments (unacceptable process continuity). Values tabulated as maximum cabin pressure reflect values at which the process was stable (i.e., no visibly broken filaments) during at least 20 minutes of continuous operation. FIG. 1 illustrates that the inventive polymers could be processed at higher cabin pressures than conventional polymers PP3155 or PP3885E1. That the effect is not simply due to the melt flow rate can be observed by comparing PP3885E1 (nominally 65 MFR) with Inventive Example 2 (nominally 55 MFR). That Inventive Example 2 runs at higher cabin pressures, produces finer fibers even though nominally lower MFR suggests the combination of polymer design features of Inventive Example 2 allows the finer fiber production. No polymers except the Inventive Examples allowed production of fibers having deniers less than 1.0 at the conditions used in this study. Note that the Inventive Example 3 (nominal 70 MFR) was not run to maximum cabin pressure due to limited material availability. Results suggest that higher cabin pressures (likely near 8000 Pa or higher) are possible with fiber sizes less than 1 denier per filament likely. It should be noted also that the calculated filament velocities (based on a combination of the mass throughput 0.47 grams per minute) and the measured fiber size (0.9 denier per filament for sample prepared using Inventive Example 2) is near 4700 m/min. This is very fast for spunbond operations using propylene polymers.

This prospective example is disclosed with reference to the catalyst synthesis methods disclosed in U.S. Pat. No. 5,891,814, dimethylsilylenebis(2-methyl-4-phenylindenyl) zirconium dichloride in toluene is combined with methylalumoxane, followed by combining with a silica oxide support to form a supported metallocene catalyst system. The metallocene catalyst is then dried and either pre-polymerized or not before adding to the polymerization system. The polymerization system comprises a 2 liter autoclave reactor flushed with N₂ while heating. Cool triethylaluminum (about 0.25 mL of a 1M solution in hexane) and the desired mM of hydrogen is then charged to the autoclave followed with about 1000 mL of propylene. The hydrogen level is adjusted such that a melt flow rate of 55 dg/min of the polypropylene homopolymer is achieved. The reactor is heated to a temperature of 50 to 80° C. The catalyst sample (about 0.075 grams bare catalyst) is loaded into an injection tube and slurried in about 2 mL of hexane. The catalyst is charged to the reactor with a flush of about 200 mL propylene to start the reaction. After about 20 minutes the reactor is cooled, vented, purged with N₂ and opened. The recovered polymer is permitted to dry in air for at least four hours then is dried for a minimum of about 2 hours at 75° C. in vacuo. After recovery the dried reactor grade polypropylene homopolymer is utilized for the fiber/fabric formation step. The polypropylene has a MFR of about 55 dg/min, a Tₘ of about 155 to 145° C. and a Mw/Mn of less than 3.0.

Spunbonding of the metallocene reactor grade homopolymer is performed on a Reicofil 4 line. The 55 dg/min reactor grade homopolymer delivery is fixed at 0.47 gram per hole per minute to form a single spunbond fabric layer. The cooling air temperature is fixed at 20° C.; the embossing roll temperature is fixed at about 137° C. and the smooth calender roll (“S-roll”) temperature is fixed at about 135° C.; calender temperature settings were from 130 to 145° C. The cabin pressure was about 7000 Pa, and the line speed was about 240 kg/hr. From this, fibers are produced having a denier of about 0.9 to 1.2 g/9000 m and the fabrics made therefrom have a basis weight of from 8 to 12 g/m² and a MD Tensile Strength of 20 to 30 N/5 cm and a CD Tensile Strength of 10 to 15 N/5 cm.

TABLE 1
Inventive Sample 1 Fiber/Fabric Production Process Conditions

| | | | Line | Cooling | Cabin | | Throughput | | | | |
|---|---|---|---|---|---|---|---|---|---|---|---|
| | | | Speed | air temp. | Pressure | Throughput | per hole | Embossing | S-roll | Calender | |
| Trial | Sample | layer | (m/min) | (C.) | (Pa) | (kg/hr) | (g/hole/min) | roll (C.) | (C.) | Settings (C.) | |
| 1 | 1 (MFR 40) | S | 240 | 20 | 1800 | 178 | 0.47 | 137 | 135 | 145 | 142 |
| 2 | 1 (MFR 40) | S | 240 | 20 | 2700 | 178 | 0.47 | 137 | 135 | 145 | 142 |
| 3 | 1 (MFR 40) | S | 240 | 20 | 5700 | 178 | 0.47 | 137 | 135 | 145 | 142 |
| 4 | 1 (MFR 40) | S | 240 | 20 | 2700 | 178 | 0.47 | 133 | 133 | 140 | 137 |
| 5 | 1 (MFR 40) | S | 240 | 20 | 2700 | 178 | 0.47 | 128 | 127 | 135 | 132 |
| 6 | 1 (MFR 40) | S | 240 | 20 | 2700 | 178 | 0.47 | 123 | 121 | 130 | 127 |

TABLE 2
Inventive Sample 2 Fiber/Fabric Production Process Conditions

| | | | Line | Cooling | Cabin | | Throughput | | | | |
|---|---|---|---|---|---|---|---|---|---|---|---|
| | | | Speed | air temp. | Pressure | Throughput | per hole | Embossing | S-roll | Calender | |
| Trial | Sample | layer | (m/min) | (C.) | (Pa) | (kg/hr) | (ghm) | roll (C.) | (C.) | Settings (C.) | |
| 7 | 2 (MFR 55) | S | 240 | 20 | 3400 | 178 | 0.47 | 123 | 120 | 128 | 125 |
| 8 | 2 (MFR 55) | S | 240 | 20 | 3400 | 178 | 0.47 | 127 | 124 | 132 | 129 |
| 9 | 2 (MFR 55) | S | 240 | 20 | 3400 | 178 | 0.47 | 130 | 128 | 135 | 132 |
| 10 | 2 (MFR 55) | S | 240 | 20 | 3400 | 178 | 0.47 | 133 | 131 | 139 | 136 |
| 11 | 2 (MFR 55) | S | 240 | 20 | 3400 | 178 | 0.47 | 136 | 135 | 144 | 141 |
| 12 | 2 (MFR 55) | S | 240 | 20 | 7000 | 178 | 0.47 | 140 | 138 | 148 | 145 |
| 13 | 2 (MFR 55) | S | 240 | 20 | 8700 | 178 | 0.47 | — | — | 148 | 145 |

TABLE 3
Inventive Sample 3 Fiber/Fabric Production Process Conditions

| | | | Line | Cooling | Cabin | | Throughput | | | | |
|---|---|---|---|---|---|---|---|---|---|---|---|
| | | | Speed | air temp. | Pressure | Throughput | per hole | Embossing | S-roll | Calender | |
| Trial | Sample | layer | (m/min) | (C.) | (Pa) | (kg/hr) | (ghm) | roll (C.) | (C.) | Settings (C.) | |
| 14 | 3 (MFR 70) | S | 240 | 20 | 4200 | 178 | 0.47 | 141 | 141 | 152 | 149 |
| 15 | 3 (MFR 70) | S | 240 | 20 | 4200 | 178 | 0.47 | 145 | 145 | 156 | 153 |
| 16 | 3 (MFR 70) | S | 240 | 20 | 4200 | 178 | 0.47 | 138 | 138 | 148 | 145 |
| 17 | 3 (MFR 70) | S | 240 | 20 | 4200 | 178 | 0.47 | 134 | 134 | 143 | 141 |
| 18 | 3 (MFR 70) | S | 240 | 20 | 4200 | 178 | 0.47 | 130 | 131 | 139 | 137 |
| 19 | 3 (MFR 70) | S | 240 | 20 | 4200 | 178 | 0.47 | 127 | 126 | 135 | 133 |
| 20 | 3 (MFR 70) | S | 240 | 20 | 4200 | 178 | 0.47 | 123 | 123 | 131 | 129 |

TABLE 4
Comparative Metallocene Sample Fiber/Fabric Production Process Conditions

| | | | Line | Cooling | Cabin | | Throughput | | | | |
|---|---|---|---|---|---|---|---|---|---|---|---|
| | | | Speed | air temp. | Pressure | Throughput | per hole | Embossing | S-roll | Calender | |
| Trial | Sample | layer | (m/min) | (C.) | (Pa) | (kg/hr) | (ghm) | roll (C.) | (C.) | Settings (C.) | |
| 21 | Achieve 3854 | S | 215 | 20 | 8000 | 184 | 0.48 | 122 | 123 | 140 | 137 |
| 22 | Achieve 3854 | S | 215 | 20 | 7500 | 184 | 0.48 | 122 | 123 | 140 | 137 |
| 23 | Achieve 3854 | S | 215 | 20 | 7000 | 184 | 0.48 | 122 | 123 | 140 | 137 |
| 24 | Achieve 3854 | S | 215 | 18/15 | 7000 | 184 | 0.48 | 122 | 123 | 140 | 137 |
| 25 | Achieve 3854 | S | 215 | 18/15 | 7500 | 184 | 0.48 | 122 | 123 | 140 | 137 |
| 26 | Achieve 3854 | S | 215 | 18/15 | 8000 | 184 | 0.48 | 122 | 123 | 140 | 137 |
| 27 | Achieve 3854 | S | 264 | 20 | 9000 | 220 | 0.58 | 123 | 122 | 140 | 137 |
| 28 | Achieve 3854 | S | 264 | 20 | 8500 | 220 | 0.58 | 122 | 123 | 140 | 137 |
| 29 | Achieve 3854 | S | 264 | 20 | 8000 | 220 | 0.58 | 121 | 123 | 140 | 137 |
| 30 | Achieve 3854 | S | 264 | 20 | 10200 | 267 | 0.70 | 120 | 124 | 140 | 137 |
| 31 | Achieve 3854 | S | 264 | 20 | 9600 | 267 | 0.70 | 119 | 124 | 140 | 137 |
| 32 | Achieve 3854 | S | 264 | 20 | 9000 | 267 | 0.70 | 118 | 125 | 140 | 137 |
| 33 | Achieve 3854 | S | 264 | 30 | 10200 | 220 | 0.58 | 121 | 122 | 140 | 137 |
| 34 | Achieve 3854 | S | 264 | 35/30 | 10200 | 220 | 0.58 | 121 | 122 | 140 | 137 |
| 35 | Achieve 3854 | S | 310 | 35/30 | 10400 | 267 | 0.70 | 121 | 122 | 140 | 137 |
| 36 | Achieve 3854 | S | 310 | 35/30 | 11000 | 267 | 0.70 | 121 | 122 | 140 | 137 |
| 37 | Achieve 3854 | SS | 390 | 20 | 2700 | 178 | 0.47 | — | — | 160 | 157 |
| 38 | Achieve 3854 | SS | 390 | 22 | 5200 | 178 | 0.47 | — | — | 160 | 157 |
| 39 | Achieve 3854 | SS | 390 | 22 | 2000 | 178 | 0.47 | — | — | 160 | 157 |
| 40 | Achieve 3854 | SS | 475 | 22 | 2700 | 178 | 0.47 | — | — | 160 | 157 |
| 41 | Achieve 3854 | SS | 550 | 22 | 5200 | 178 | 0.47 | — | — | 160 | 157 |
| 42 | Achieve 3854 | SS | 390 | 22 | 2000 | 178 | 0.47 | 149 | 151 | 164 | 161 |
| 43 | Achieve 3854 | SS | 475 | 22 | 2700 | 178 | 0.47 | — | — | 164 | 161 |
| 44 | Achieve 3854 | SS | 550 | 22 | 5200 | 178 | 0.47 | — | — | 164 | 161 |
| 45 | Achieve 3854 | SS | 390 | 22 | 2000 | 178 | 0.47 | 143 | 144 | 156 | 153 |
| 46 | Achieve 3854 | SS | 475 | 22 | 2700 | 178 | 0.47 | — | — | 156 | 153 |
| 47 | Achieve 3854 | SS | 550 | 22 | 5200 | 178 | 0.47 | — | — | 156 | 153 |
| 48 | Achieve 3854 | SS | 390 | 22 | 2000 | 178 | 0.47 | 140 | 140 | 152 | 149 |
| 49 | Achieve 3854 | SS | 475 | 22 | 2700 | 178 | 0.47 | — | — | 152 | 149 |
| 50 | Achieve 3854 | SS | 550 | 22 | 5200 | 178 | 0.47 | — | — | 152 | 149 |
| 51 | Achieve 3854 | S | 238 | 22 | 2700 | 178 | 0.47 | 144 | 145 | 156 | 153 |

TABLE 5
Comparative Ziegler-Natta Sample Fiber/Fabric Production Process Conditions

| | | | Line | Cooling | Cabin | | Throughput | | | | |
|---|---|---|---|---|---|---|---|---|---|---|---|
| | | | Speed | air temp. | Pressure | Throughput | per hole | Embossing | S-roll | Calender | |
| Trial | Sample | layer | (m/min) | (C.) | (Pa) | (kg/hr) | (ghm) | roll (C.) | (C.) | Settings (C.) | |
| 52 | PP3155 | S | 215 | 15 | 4000 | 184 | 0.48 | 122 | 122 | 140 | 137 |
| 53 | PP3155 | S | 215 | 15 | 3500 | 184 | 0.48 | 122 | 122 | 140 | 137 |
| 54 | PP3155 | S | 215 | 15 | 3000 | 184 | 0.48 | 122 | 122 | 140 | 137 |
| 55 | PP3155 | S | 215 | 20 | 3000 | 184 | 0.48 | 122 | 122 | 140 | 137 |
| 56 | PP3155 | S | 215 | 20 | 3500 | 184 | 0.48 | 122 | 122 | 140 | 137 |
| 57 | PP3155 | S | 215 | 20 | 4000 | 184 | 0.48 | 122 | 122 | 140 | 137 |
| 58 | PP3155 | S | 264 | 20 | 4800 | 225 | 0.59 | 122 | 122 | 140 | 137 |
| 59 | PP3155 | S | 264 | 20 | 4400 | 225 | 0.59 | 122 | 122 | 140 | 137 |
| 60 | PP3155 | S | 264 | 20 | 4000 | 225 | 0.59 | 122 | 122 | 140 | 137 |
| 61 | PP3155 | S | 310 | 20 | 5600 | 266 | 0.70 | 122 | 122 | 140 | 137 |
| 62 | PP3155 | S | 310 | 20 | 5200 | 266 | 0.70 | 122 | 122 | 140 | 137 |
| 63 | PP3155 | S | 310 | 20 | 4800 | 266 | 0.70 | 122 | 122 | 140 | 137 |
| 64 | PP3155 | S | — | — | — | — | — | — | — | — | — |
| 65 | PP3155 | S | 182 | 20 | 3500 | 178 | 0.47 | — | — | 131 | 129 |
| 66 | PP3155 | S | 240 | 25 | 5000 | 178 | 0.47 | 139 | 138 | 148 | 145 |
| 67 | PP3155 | S | 240 | 25 | 5000 | 178 | 0.47 | 143 | 125 | 153 | 150 |
| 68 | PP3155 | S | 240 | 25 | 5000 | 178 | 0.47 | 147 | 146 | 158 | 155 |
| 69 | PP3155 | S | 295 | 25 | 5600 | 218 | 0.57 | 139 | 138 | 148 | 145 |
| 70 | PP3155 | S | 295 | 25 | 5600 | 218 | 0.57 | 144 | 144 | 153 | 150 |
| 71 | PP3155 | S | 295 | 25 | 5600 | 218 | 0.57 | 134 | 134 | 143 | 140 |

TABLE 6
Comparative Ziegler-Natta Sample Fiber/Fabric Production Process Conditions

| | | | Line | Cooling | Cabin | | Throughput | | | | |
|---|---|---|---|---|---|---|---|---|---|---|---|
| Trial | | | Speed | air temp. | Pressure | Throughput | per hole | Embossing | S-roll | Calender | |
| ref. | Sample | layer | (m/min) | (C.) | (Pa) | (kg/hr) | (ghm) | roll (C.) | (C.) | Settings (C.) | |
| 72 | PP3885E1 | S | 215 | 15 | 8000 | 179 | 0.47 | 123 | 123 | 140 | 137 |
| 73 | PP3885E1 | S | 215 | 15 | 7500 | 179 | 0.47 | 123 | 123 | 140 | 137 |
| 74 | PP3885E1 | S | 215 | 15 | 7000 | 179 | 0.47 | 123 | 123 | 140 | 137 |
| 75 | PP3885E1 | S | 215 | 20 | 7000 | 179 | 0.47 | 123 | 123 | 140 | 137 |
| 76 | PP3885E1 | S | 215 | 20 | 7500 | 179 | 0.47 | 123 | 123 | 140 | 137 |
| 77 | PP3885E1 | S | 215 | 20 | 8000 | 179 | 0.47 | 123 | 122 | 140 | 137 |
| 78 | PP3885E1 | S | 238 | 20 | — | 180 | 0.47 | 146 | 147 | 156 | 153 |

TABLE 7
Inventive Example 1 Fiber/Fabric Properties

| | | | Basis | | | | | |
|---|---|---|---|---|---|---|---|---|
| Trial | | | Weight | | MD | CD | MD | CD |
| ref. | Sample | layer | (g/m²) | Denier | strength | strength | elongation | elongation |
| 1 | 1 (MFR 40) | S | 12 | 1.95 | 10 | 7 | 22 | 32 |
| 2 | 1 (MFR 40) | S | 12 | 1.36 | 22 | 12 | 39 | 41 |
| 3 | 1 (MFR 40) | S | 12 | 1.05 | 43 | 19 | 52 | 60 |
| 4 | 1 (MFR 40) | S | 12 | — | 22 | 13 | 46 | 48 |
| 5 | 1 (MFR 40) | S | 12 | — | 25 | 12 | 49 | 47 |
| 6 | 1 (MFR 40) | S | 12 | — | 21 | 11 | 42 | 44 |

TABLE 8
Inventive Example 2 Fiber/Fabric Properties

| | | | Basis | | | | | |
|---|---|---|---|---|---|---|---|---|
| Trial | | | Weight | | MD | CD | MD | CD |
| ref. | Sample | layer | (g/m²) | Denier | strength | strength | elongation | elongation |
| 7 | 2 (MFR 55) | S | 12 | 1.3 | 25 | 13 | 48 | 49 |
| 8 | 2 (MFR 55) | S | 12 | — | 24 | 14 | 46 | 51 |
| 9 | 2 (MFR 55) | S | 12 | — | 26 | 14 | 45 | 49 |
| 10 | 2 (MFR 55) | S | 12 | — | 29 | 13 | 44 | 4 |
| 11 | 2 (MFR 55) | S | 12 | — | 22 | 13 | 32 | 43 |
| 12 | 2 (MFR 55) | S | 12 | 0.99 | 45 | 19 | 46 | 51 |
| 13 | 2 (MFR 55) | S | 12 | 0.85 | 45 | 20 | 41 | 55 |

TABLE 9
Inventive Example 3 Fiber/Fabric Properties

| | | | Basis | | | | | |
|---|---|---|---|---|---|---|---|---|
| | | | Weight | | MD | CD | MD | CD |
| Trial | Sample | layer | (g/m²) | Denier | strength | strength | elongation | elongation |
| 14 | 3 (MFR 70) | S | 12 | 1.32 | 16 | 10 | 21 | 32 |
| 15 | 3 (MFR 70) | S | 12 | — | 12 | 8 | 14 | 22 |
| 16 | 3 (MFR 70) | S | 12 | — | 22 | 12 | 31 | 34 |
| 17 | 3 (MFR 70) | S | 12 | — | 25 | 13 | 36 | 40 |
| 18 | 3 (MFR 70) | S | 12 | — | 28 | 13 | 42 | 40 |
| 19 | 3 (MFR 70) | S | 12 | — | 28 | 16 | 44 | 52 |
| 20 | 3 (MFR 70) | S | 12 | — | 28 | 14 | 49 | 44 |

TABLE 10
Comparative Metallocene Example Fiber/Fabric Properties

| | | | Basis Weight | | MD | CD | MD | CD |
|---|---|---|---|---|---|---|---|---|
| Trial | Sample | layer | (g/m²) | Denier | strength | strength | elongation | elongation |
| 21 | Achieve 3854 | S | 13.0 | 1.19 | 52 | 19 | 53 | 51 |
| 22 | Achieve 3854 | S | 13.0 | 1.09 | 50 | 19 | 50 | 54 |
| 23 | Achieve 3854 | S | 13.0 | 1.23 | 44 | 18 | 47 | 47 |
| 24 | Achieve 3854 | S | 13.0 | 1.24 | 47 | 18 | 49 | 52 |
| 25 | Achieve 3854 | S | 13.0 | 1.16 | 49 | 19 | 49 | 48 |
| 26 | Achieve 3854 | S | 13.0 | 1.19 | 53 | 19 | 51 | 53 |
| 27 | Achieve 3854 | S | 13.0 | 1.33 | 48 | 16 | 45 | 45 |
| 28 | Achieve 3854 | S | 13.0 | 1.26 | 46 | 15 | 46 | 46 |
| 29 | Achieve 3854 | S | 13.0 | 1.24 | 43 | 16 | 42 | 45 |
| 30 | Achieve 3854 | S | 13.0 | 1.41 | 42 | 13 | 39 | 40 |
| 31 | Achieve 3854 | S | 13.0 | 1.43 | 38 | 12 | 36 | 38 |
| 32 | Achieve 3854 | S | 13.0 | 1.55 | 33 | 11 | 33 | 34 |
| 33 | Achieve 3854 | S | 13.0 | 1.09 | 44 | 14 | 37 | 37 |
| 34 | Achieve 3854 | S | 13.0 | 1.09 | 45 | 15 | 41 | 39 |
| 35 | Achieve 3854 | S | 13.0 | 1.17 | 36 | 12 | 32 | 35 |
| 36 | Achieve 3854 | S | 13.0 | 1.30 | 36 | 11 | 32 | 36 |
| 37 | Achieve 3854 | SS | 14 | 1.5 | 33 | 17 | 46 | 42 |
| 38 | Achieve 3854 | SS | 14 | 1.2 | 62 | 26 | 63 | 61 |
| 39 | Achieve 3854 | SS | 14 | 1.9 | 20 | 13 | 32 | 42 |
| 40 | Achieve 3854 | SS | 12 | 0.81 | 28 | 12 | 42 | 47 |
| 41 | Achieve 3854 | SS | 10 | 0.61 | 39 | 17 | 51 | 64 |
| 42 | Achieve 3854 | SS | 14 | — | 18 | 12 | 31 | 40 |
| 43 | Achieve 3854 | SS | 12 | — | 24 | 10 | 35 | 38 |
| 44 | Achieve 3854 | SS | 10 | — | 35 | 17 | 45 | 56 |
| 45 | Achieve 3854 | SS | 14 | — | 27 | 14 | 48 | 49 |
| 46 | Achieve 3854 | SS | 12 | — | 28 | 12 | 50 | 46 |
| 47 | Achieve 3854 | SS | 10 | — | 37 | 16 | 47 | 64 |
| 48 | Achieve 3854 | SS | 14 | — | 24 | 13 | 42 | 42 |
| 49 | Achieve 3854 | SS | 12 | — | 27 | 13 | 48 | 53 |
| 50 | Achieve 3854 | SS | 10 | — | 36 | 15 | 52 | 57 |
| 51 | Achieve 3854 | S | 12 | — | 16 | 7 | 20 | 24 |

TABLE 11
Comparative Zeigler-Natta Example Fiber/Fabric Properties

| | | | Basis | | | | | |
|---|---|---|---|---|---|---|---|---|
| | | | Weight | | MD | CD | MD | CD |
| Trial | Sample | layer | (g/m²) | Denier | strength | strength | elongation | elongation |
| 52 | PP3155 | S | 13.0 | 1.32 | 40 | 15 | 62 | 57 |
| 53 | PP3155 | S | 13.0 | 1.37 | 34 | 15 | 63 | 67 |
| 54 | PP3155 | S | 13.0 | 1.49 | 37 | 15 | 64 | 72 |
| 55 | PP3155 | S | 13.0 | 1.57 | 35 | 13 | 66 | 62 |
| 56 | PP3155 | S | 13.0 | 1.35 | 35 | 14 | 46 | 50 |
| 57 | PP3155 | S | 13.0 | 1.43 | 38 | 15 | 60 | 67 |
| 58 | PP3155 | S | 12.6 | 1.39 | 34 | 13 | 55 | 64 |
| 59 | PP3155 | S | 12.6 | 1.43 | 37 | 11 | 60 | 53 |
| 60 | PP3155 | S | 12.6 | 1.47 | 33 | 12 | 58 | 58 |
| 61 | PP3155 | S | 12.6 | 1.53 | 28 | 11 | 64 | 51 |
| 62 | PP3155 | S | 12.6 | 1.35 | 28 | 10 | 38 | 43 |
| 63 | PP3155 | S | 12.6 | 1.41 | 24 | 9 | 41 | 41 |
| 64 | PP3155 | S | — | — | — | — | — | — |
| 65 | PP3155 | S | 15 | — | — | — | — | — |
| 66 | PP3155 | S | 12 | 1.2 | 34 | 19 | 70 | 78 |
| 67 | PP3155 | S | 12 | — | 33 | 20 | 63 | 75 |
| 68 | PP3155 | S | 12 | — | 28 | 16 | 49 | 58 |
| 69 | PP3155 | S | 12 | 1.35 | 31 | 17 | 75 | 77 |
| 70 | PP3155 | S | 12 | — | 30 | 17 | 64 | 73 |
| 71 | PP3155 | S | 12 | — | 31 | 16 | 77 | 68 |

TABLE 12
Comparative Ziegler-Natta Example Fiber/Fabric Properties

| | | | Basis | | | | | |
|---|---|---|---|---|---|---|---|---|
| | | | Weight | | MD | CD | MD | CD |
| Trial | Sample | layer | (g/m²) | Denier | strength | strength | elongation | elongation |
| 72 | PP3885E1 | S | 13.0 | 1.12 | 39 | 19 | 77 | 71 |
| 73 | PP3885E1 | S | 13.0 | 1.16 | 38 | 19 | 77 | 81 |
| 74 | PP3885E1 | S | 13.0 | 1.16 | 38 | 18 | 84 | 79 |
| 75 | PP3885E1 | S | 13.0 | 1.02 | 38 | 17 | 83 | 73 |
| 76 | PP3885E1 | S | 13.0 | 1.12 | 36 | 18 | 70 | 77 |
| 77 | PP3885E1 | S | 13.0 | 1.26 | 38 | 18 | 74 | 78 |
| 78 | PP3885E1 | S | 12 | — | 26 | 16 | 40 | 54 |

TABLE 13
Cabin Pressure Influence

| Press. | Sample 2 | Sample 3 |
|---|---|---|
| 1800 | — | — |
| 2700 | — | — |
| 5700 | — | — |
| 3400 | 1.3 | — |
| 7000 | 1 | — |
| 8700 | 0.9 | — |
| 4200 | — | 1.3 |
| 3000 | — | — |
| 3500 | — | — |
| 4000 | — | — |
| 5000 | — | — |
| 8000 | — | — |
| 7500 | — | — |
| 7000 | — | — |

TABLE 14
Tensile Strength as a function of Denier of the Fiber

| | | Tensile, MD N/5 cm | Tensile, MD N/5 cm |
|---|---|---|---|
| | denier | Sample 2 | Sample 3 |
| 1 | 1.95 | — | — |
| 2 | 1.36 | — | — |
| 3 | 1.05 | — | — |
| 11 | 1.3 | 22 | — |
| 12 | 0.99 | 45 | — |
| 13 | 0.85 | 45 | — |
| 16 | 1.3 | — | 22 |
| 66 | 1.2 | — | — |

Having described the various descriptive elements and numerical ranges to elucidate preferred embodiments of the propylene-based fibers, fabrics and laminates that comprise such, described here in numbered embodiments is:

A nonwoven fabric of fibers comprising one or more primary polypropylenes having a molecular weight distribution of less than 3.5 and a melt flow rate within the range from 5 to 500 dg/min, the fibers having an average diameter of less than 20 μm, or a denier (g/9000 m) of less than 2.0, or both.

1. The propylene-based fabric of numbered embodiment 1, wherein the fabrics have a MD Tensile Strength (WSP 110.4 (05)) of greater than 20 N/5 cm when calendered at a temperature within the range from 110 to 150° C.
2. The propylene-based fabric of numbered embodiment 1 or 2, wherein the fabrics have a CD Tensile Strength (WSP 110.4 (05)) of greater than 10 N/5 cm when calendered at a temperature within the range from 110 to 150° C.
3. The propylene-based fabric of any one of the previously numbered embodiments, wherein the primary polypropylene comprises from 0 to 5 wt % comonomer derived units selected from ethylene and C4 to C12 α-olefins.
4. The propylene-based fabric of any one of the previously numbered embodiments, wherein the primary polypropylene is reactor grade.
5. The propylene-based fabric of any one of the previously numbered embodiments, wherein the primary polypropylene has a melting point of less than 165° C.
6. The propylene-based fabric of any one of the previously numbered embodiments, wherein the primary polypropylene is produced by a single-site catalyst system.
7. The propylene-based fabric of any one of the previously numbered embodiments, wherein the primary polypropylene has from 0.1 to 15 mole % regio-defects (¹³C NMR).
8. The propylene-based fabric of any one of the previously numbered embodiments, wherein the fabric is a spunbond fabric.
9. The propylene-based fabric of any one of the previously numbered embodiments, wherein the fabric has an average pore size within the range of from 10 to 200 μm.
10. The propylene-based fabric of any one of the previously numbered embodiments, wherein the fabric has basis weight of from less than 14 g/m².
11. A laminate comprising one or more layers of the propylene-based fabric of any one of the previously numbered embodiments.
12. The laminate of embodiment 12 wherein the laminate comprises one or more secondary layers comprising other fabrics, nets, coform fabrics, scrims, and/or films prepared from natural and/or synthetic materials.
13. The laminate of embodiment 13, wherein the one or more secondary layers comprise materials selected from the group consisting of primary polypropylene, polyethylene, plastomers, polyurethane, polyester, styrenic block copolymers, ethylene vinyl acetate copolymers, polyamide, polycarbonate, cellulosics (e.g., cotton, Rayon™, Lyocell™, Tencil™), wood, viscose, and blends of any two or more of these materials.
14. The laminate of embodiment 12, wherein the propylene-based nonwoven fabric is characterized by the designation “P”, the laminate is selected from structures consisting of MP, MPM, PP, PPP, PPPP, PPM, PMP, PMMP, PPMPP, PMMPP, PMPPP, PPMMPP, PMPMP, PPPMPP, SP, SPS, PP, PPP, PPPP, DPPPP, MPPPP, SPPPP, PPS, PSP, PSSP, PPSPP, PSSPP, PSPPP, PPSSPP, PSPSP, PPPSPP, DP, DDP, DPD, DPP, DDDDP, PPD, PDP, PDDP, PPDPP, PDDPP, PPDDPP, DMP, DDMPP, PDMDP, DPMPD, DDPMPD, DDPMPDD, DDPMMPDD, DPMMPD, PDMDMD, PMDMP, PDMMDD, PPDMDPP, DDDDMP, PPDMMDPP, FP, MPF, FPP, FPPP, FPPPP, FPPF, FPFPF, FPPM, PFP, PMFP, PPFPP, PFFPP, PMFPP, PPMFPP, PFD, PDFD, PDDFFD, PDFDD, DPF, DFP, DDDFP, FDP, PDDF, PFDPP, FPDDPP, PFDDPP, DMPF, DFMPP, PDFDP, DPFPD, DDPFPD, DDPFPDD, DDPFFPDD, DPFFPD, PFDFD, PFDFP, SFMP, SSFMP, and PFFP, wherein “M” represents meltblown fabric layers, “S” represents spunbond fabric layers, “F” represents film layers, and “D” represents dry-laid (carded or air-laid) fabric layers.
15. An absorbent or barrier product made from the fabric of any one of the previously numbered embodiments, the articles comprising personal care products, baby diapers, training pants, absorbent underpads, swim wear, wipes, feminine hygiene products, bandages, wound care products, medical garments, surgical gowns, filters, adult incontinence products, surgical drapes, coverings, garments, and cleaning articles and apparatus.
16. A method of forming a fabric comprising meltspinning one or more primary polypropylenes of any one of the previously numbered embodiments at fiber forming velocities of greater than 3000 m/min to produce fibers having an average diameter of less than 20 μm, or an denier (g/9000 m) of less than 2.0.
17. The method of embodiment 17, wherein the meltspun process is a spunbond process and the extruded fibers are exposed to an attenuating air pressure of greater than 2000 Pa.
18. The method of any one of embodiments 17-18, wherein the fabrics are calendered at a temperature within the range from 110 to 150° C. and have a MD Tensile Strength (WSP 110.4 (05)) of greater than 20 N/5 cm.
19. The method of any one of embodiments 17-19, wherein the fabrics are calendered at a temperature within the range from 110 to 150° C. and have a CD Tensile Strength (WSP 110.4 (05)) of greater than 10 N/5 cm.
20. The method of any one of embodiments 17-20, wherein the primary polypropylene is produced by a single-site catalyst system.
21. The method of any one of embodiments 17-21, wherein the spunbond line throughput is within the range from 150 to 300 kg/hr.
22. The method of any one of embodiments 17-22, wherein the spunbond line throughput per hole is within the range from 0.30 to 0.90 ghm.

1. A nonwoven fabric of fibers comprising one or more primary polypropylenes having a molecular weight distribution of less than 3.5 and a melt flow rate within the range from 5 to 500 dg/min, the fibers having at least one of an average diameter of less than 20 μm or a denier (g/9000 m) of less than 2.0.

2. The nonwoven fabric of claim 1, wherein the fabrics have a MD Tensile Strength (WSP 110.4 (05)) of greater than 20 N/5 cm when calendered at a temperature within the range from 110 to 150° C.

3. The nonwoven fabric of claim 1, wherein the fabrics have a CD Tensile Strength (WSP 110.4 (05)) of greater than 10 N/5 cm when calendered at a temperature within the range from 110 to 150° C.

4. The nonwoven fabric of claim 1, wherein the primary polypropylene comprises from 0 to 5 wt % comonomer derived units selected from ethylene and C4 to C12 α-olefins.

5. The nonwoven fabric of claim 1, wherein the primary polypropylene is reactor grade.

6. The nonwoven fabric of claim 1, wherein the primary polypropylene has a melting point of less than 165° C.

7. The nonwoven fabric of claim 1, wherein the primary polypropylene is produced by a single-site catalyst system.

8. The nonwoven fabric of claim 1, wherein the primary polypropylene has from 0.1 to 15 mole % regio-defects (¹³C NMR).

9. The nonwoven fabric of claim 1, wherein the fabric is a spunbond fabric.

10. The nonwoven fabric of claim 1, wherein the fabric has an average pore size within the range of from 10 to 200 μm.

11. The nonwoven fabric of claim 1, wherein the fabric has basis weight of from less than 14 g/m².

12. A laminate comprising one or more layers of the nonwoven fabric of claim 1.

13. The laminate of claim 12, wherein the laminate comprises one or more secondary layers comprising other fabrics, nets, coform fabrics, scrims, and/or films prepared from natural and/or synthetic materials.

14. The laminate of claim 13, wherein the one or more secondary layers comprise materials selected from the group consisting of primary polypropylene, polyethylene, plastomers, polyurethane, polyester, styrenic block copolymers, ethylene vinyl acetate copolymers, polyamide, polycarbonate, cellulosics (e.g., cotton, Rayon™, Lyocell™, Tencil™), wood, viscose, and blends of any two or more of these materials.

15. The laminate of claim 12, wherein the propylene-based nonwoven fabric is characterized by the designation “P”, the laminate is selected from structures consisting of MP, MPM, PP, PPP, PPPP, PPM, PMP, PMMP, PPMPP, PMMPP, PMPPP, PPMMPP, PMPMP, PPPMPP, SP, SPS, PP, PPP, PPPP, DPPPP, MPPPP, SPPPP, PPS, PSP, PSSP, PPSPP, PSSPP, PSPPP, PPSSPP, PSPSP, PPPSPP, DP, DDP, DPD, DPP, DDDDP, PPD, PDP, PDDP, PPDPP, PDDPP, PPDDPP, DMP, DDMPP, PDMDP, DPMPD, DDPMPD, DDPMPDD, DDPMMPDD, DPMMPD, PDMDMD, PMDMP, PDMMDD, PPDMDPP, DDDDMP, PPDMMDPP, FP, MPF, FPP, FPPP, FPPPP, FPPF, FPFPF, FPPM, PFP, PMFP, PPFPP, PFFPP, PMFPP, PPMFPP, PFD, PDFD, PDDFFD, PDFDD, DPF, DFP, DDDFP, FDP, PDDF, PFDPP, FPDDPP, PFDDPP, DMPF, DFMPP, PDFDP, DPFPD, DDPFPD, DDPFPDD, DDPFFPDD, DPFFPD, PFDFD, PFDFP, SFMP, SSFMP, and PFFP, wherein “M” represents meltblown fabric layers, “S” represents spunbond fabric layers, “F” represents film layers, and “D” represents dry-laid (carded or air-laid) fabric layers.

16. An absorbent or barrier product made from the nonwoven fabric of claim 1, the articles comprising personal care products, baby diapers, training pants, absorbent underpads, swim wear, wipes, feminine hygiene products, bandages, wound care products, medical garments, surgical gowns, filters, adult incontinence products, surgical drapes, coverings, garments, and cleaning articles and apparatus.

17. A method of forming a fabric comprising meltspinning one or more primary polypropylenes of claim 1 at fiber forming velocities of greater than 3000 m/min to produce fibers having an average diameter of less than 20 μm, or an denier (g/9000 m) of less than 2.0.

18. The method of claim 17, wherein the meltspun process is a spunbond process and the extruded fibers are exposed to an attenuating air pressure of greater than 2000 Pa.

19. The method of claim 17, wherein the is calendered at a temperature within the range from 110 to 150° C. and have a MD Tensile Strength (WSP 110.4 (05)) of greater than 20 N/5 cm.

20. The method of claim 17, wherein the is calendered at a temperature within the range from 110 to 150° C. and have a CD Tensile Strength (WSP 110.4 (05)) of greater than 10 N/5 cm.

21. The method of claim 17, wherein the primary polypropylene is produced by a single-site catalyst system.

22. The method of claim 17, wherein the spunbond line throughput is within the range from 150 to 300 kg/hr.

23. The method of claim 17, wherein the spunbond line throughput per hole is within the range from 0.30 to 0.90 ghm.
